(19) United States
(12) Patent Application Publication
(10) Pub. No.: US 2002/0032160 A1
(43) Pub. Date: Mar. 14, 2002

(54) Compositions & formulations with an epiandrosterone or a ubiquinone & kits & their use for treatment of asthma symptoms & for reducing adenosine/adenosine receptor levels

(76) Inventor: Jonathan W. Nyce, Titusville, NJ (US)

Correspondence Address: EPIGENESIS PHARMACEUTICALS, 7 CLARK DRIVE, CANBURY, NJ, 08520, US

(21) Appl. No.: 09/841,426

(22) Filed: Apr. 24, 2001

Related U.S. Application Data
(63) Continuation-in-part of application No. 09/488,236, filed on Jan. 20, 2000.
Application No. 09/488,236 is a continuation of application No. 08/861,962, filed on May 22, 1997, now Pat. No. 6,087,351.
Application No. 08/861,962 is a division of application No. 08/393,863, filed on Feb. 24, 1995, now Pat. No. 5,660,835.

(51) Int. Cl. A61K 31/704; A61K 31/66; A61K 31/56

(52) U.S. Cl. 514/26; 514/129; 514/178

(57) ABSTRACT

A composition and various formulations comprise preventative or therapeutic amounts of an epiandrosterone, analogue thereof or salt thereof, and/or a ubiquinone or salt thereof, and a pharmaceutically or veterinarily acceptable carrier or diluent. The composition and formulations are useful for treating bronchoconstriction, respiratory tract inflammation and allergies, asthma, and cancer. A method of treating diseases associated with low adenosine levels or adenosine depletion comprises administering folinic acid or a pharmaceutically acceptable salt hereof in a preventative or therapeutic amount, or an amount effective to treat adenosine depletion.

### RELATED APPLICATIONS

This is a continuation-in-part of U.S. patent application Ser. No. 09/488,236, filed Jan. 20, 2000 and now pending; which is a continuation of U.S. patent application Ser. No. 08/861,962, filed May 22, 1997 and now U.S. Pat. No. 6,087,351; which is a divisional of U.S. patent application Ser. No. 08/393,863, filed Feb. 24, 1995, now U.S. Pat. No. 5,660,835, all by the present inventor.

The work leading to this invention was made at least in part with U.S. Government support under National Cancer Institute Grant No. CA7217. The U.S. Government may have certain rights in the invention.

### FIELD OF THE INVENTION

This invention concerns itself with a method of treating bronchoconstriction, lung inflammation and allergies, asthma, and cancer by administering an epiandrosterone, analogs thereof, a ubiquinone, and/or their pharmaceutically acceptable salts. This invention also concerns itself with a method of treating adenosine depletion by administration of folinic acid or a pharmaceutically acceptable salt thereof.

### DESCRIPTION OF THE BACKGROUND

Adenosine is a purine that contributes to intermediary metabolism and participates in the regulation of physiological activity in a variety of mammalian tissues. Adenosine participates in many local regulatory mechanisms, such as those occurring in synapses in the central nervous system (CNS) and at neuroeffector junctions in the peripheral nervous system. In the CNS, adenosine inhibits the release of a variety of neurotransmitters, such as acetylcholine, noradrenaline, dopamine, serotonin, glutamate, and GABA; depresses neurotransmission; reduces neuronal firing to induce spinal analgesia and possesses anxiolytic properties. In the heart, adenosine suppresses pacemaker activity, slows AV conduction, possesses antiarrhythmic and arrhythmogenic effects, modulates autonomic control and triggers the synthesis and release of prostaglandins. In addition, adenosine has potent vasodilatory effects and modulates vascular tone. Adenosine is currently being used clinically for the treatment of super ventricular tachycardia and other cardia anomalies. Adenosine analogues also are being investigated for use as anticonvulsant, anxiolytic and neuro protective agents. Adenosine has also been implicated as a primary determinant underlying the symptoms of bronchial asthma and other respiratory diseases, the induction of bronchoconstriction and the contraction of airway smooth muscle. Moreover, adenosine causes bronchoconstriction in asthmatics but not in non-asthmatics. Other experimental data suggest the possibility that adenosine receptors may also be involved in allergic and inflammatory responses. It has been postulated that the modulation of signal transduction at the surface of inflammatory cells influences acute inflammation. Adenosine is said to inhibit the production of super-oxide by stimulated neutrophils. Moreover, the treatment of experimental allergic uveitis produced a marked reduction in inflammation. Adenosine may attenuate this behavior by reducing the hyperactivity of the central dopaminergic system.

Diseases and conditions, such as asthma, are common diseases in industrialized countries, and in the United States alone account for extremely high health care costs. These diseases or conditions have recently been increasing at an alarming rate, both in terms of prevalence, morbidity and mortality. In spite of this, their underlying causes still remain poorly understood.

Dehydroepiandrosterone (DHEA) is a naturally occurring steroid secreted by the adrenal cortex with apparent chemoprotective properties. Epidemiological studies have shown that low endogenous levels of DHEA correlate with increased risk of developing some forms of cancer, such as pre-menopausal breast cancer in women and bladder cancer in both sexes. The ability of DHEA, DHEA analogues and their salts to inhibit carcinogenesis is believed to result from their uncompetitive inhibition of the activity of the enzyme glucose 6-phosphate dehydrogenase (G6PDH). G6PDH is the rate limiting enzyme of the hexose monophosphate pathway, a major source of intracellular ribose-5-phosphate and NADPH. Ribose-5 phosphate is a necessary substrate for the synthesis of both ribo- and deoxyribonucleotides required for the synthesis of RNA and DNA. NADPH is a cofactor also involved in nucleic acid biosynthesis and the synthesis of hydroxmethylglutaryl Coenzyme A reductase (HMG CoA reductase). HMG CoA reductase is an unusual enzyme that requires two moles of NADPH for each mole of product, mevalonate, produced. Thus, it appears that HMG CoA reductase would be ultra sensitive to DHEA-mediated NADPH depletion, and that DHEA-treated cells would rapidly show the depletion of intracellular pools of mevalonate. Mevalonate is required for DNA synthesis, and DHEA arrests human cells in the GI phase of the cell cycle in a manner closely resembling that of the direct HMG CoA. Because G6PDH produces mevalonic acid used in cellular processes such as protein isoprenylation and the synthesis of dolichol, a precursor for glycoprotein biosynthesis, DHEA inhibits carcinogenesis by depleting mevalonic acid and thereby inhibiting protein isoprenylation and glycoprotein synthesis. Mevalonate is the central precursor for the synthesis of cholesterol, as well as for the synthesis of a variety of non-sterol compounds involved in post-translational modification of proteins such as farnesyl pyrophosphate and geranyl pyrophosphate; for dolichol, which is required for the synthesis of glycoproteins involved in cell-to-cell communication and cell structure; and for ubiquinone, an anti-oxidant with an established role in cellular respiration. It has long been known that patients receiving steroid hormones of adrenocortical origin at pharmacologically appropriate doses show increased incidence of infectious disease.

DHEA, also known as 3β-hydroxyandrost-5-en-17-one or dehydroiso-androsterone, is a 17-ketosteroid which is quantitatively one of the major adrenocortical steroid hormones found in mammals. Although DHEA appears to serve as an intermediary in gonadal steroid synthesis, the primary physiological function of DHEA has not been fully understood. It has been known, however, that levels of this hormone begin to decline in the second decade of life, reaching 5% of the original level in the elderly.) Clinically, DHEA has been used systemically and/or topically for treating patients suffering from psoriasis, gout, hyperlipemia, and it has been administered to post-coronary patients. In mammals, DHEA has been shown to have weight optimizing and anti-carcinogenic effects, and it has been used clinically in Europe in conjunction with estrogen as an agent to reverse menopausal symptoms and also has been used in the treatment of manic depression, schizophrenia, and Alzheimer's disease. DHEA has also been used clinically at 40 mg/kg/day in the treatment of advanced cancer and multiple sclerosis. Mild androgenic effects, hirsutism, and increased libido were the side effects observed. These side effects can be overcome by monitoring the dose and/or by using analogues. The subcutaneous or oral administration of DHEA to improve the host's response to infections is known, as is the use of a patch to deliver DHEA. DHEA is also known as a precursor in a metabolic pathway which ultimately leads to more powerful agents that increase immune response in mammals. That is, DHEA acts as a biphasic compound: it acts as an immuno-modulator when converted to androstenediol or androst-5-ene-3β,17β-diol (βAED), or androstenetriol or androst-5-ene-3β,7β,17β-triol (βAET). However, in vitro DHEA has certain lymphotoxic and suppressive effects on cell proliferation prior to its conversion to βAED and/or βAET. It is, therefore, believed that the superior immunity enhancing properties obtained by administration of DHEA result from its conversion to more active metabolites.

Adequate ubiquinone levels have been found to be essential for maintaining proper cardiac function, and the administration of exogenous ubiquinone has recently been shown to have beneficial effect in patients with chronic heart failure. Ubiquinone depletion has been observed in humans and animals treated with lovastatin, a direct HMG CoA reductase inhibitor. Such lovastatin-induced depletion of ubiquinone has been shown to lead to chronic heart failure, or to a shift from low heart failure into life-threatening high grade heart failure. DHEA, unlike lovastatin, inhibits HMG CoA reductase indirectly by inhibiting G6PDH and depleting NADPH, a required cofactor for HMG CoA reductase. However, DHEA's indirect inhibition of HMG CoA reductase suffices to deplete intracellular mevalonate. This effect adds to the depletion of ubiquinone, and may result in chronic heart failure following long term usage. Thus, although DHEA was once considered a safe drug, it is now predicted that with long term administration of DHEA or its analogues, chronic heart failure may occurs as a complicating side effect. Further, some analogues of DHEA produce this side effect to a greater extent because, in general, they are more potent inhibitors of G6PDH than DHEA.

Folinic acid is an intermediate product of the metabolism of folic acid; the active form into which that acid is converted in the body. Ascorbic acid is required as a necessary factor in the conversion process. Folinic acid has been used therapeutically as an antidote to folic acid antagonists such as methotrexate which block the conversion of folic acid into folinic acid. Additionally, folinic acid has been used as an anti-anemic (combating folate deficiency). The use of folinic acid in patients afflicted with adenosine depletion, or in a method to therapeutically elevate adenosine levels in the brain or other organ, has heretofore neither been suggested nor described.

In view of the foregoing, it is readily apparent that (i) methods of inducing adenosine depletion may be useful in treating respiratory and airway conditions such as asthma, surfactant depletion, bronchoconstriction, lung inflammation, and allergies; and (ii) adenosine depletion may lead to a broad variety of deleterious conditions, and that methods of treating adenosine depletion and those conditions may be an extremely useful means of therapeutic intervention.

The population of the U.S. and of the world in general living longer lives, many of these diseases and conditions have become more prevalent given the more advanced age of this segment of the population, and would benefit from new products and preventative and therapeutic treatments. The availability of a novel strategy to prevent and/or treat disorders such as bronchoconstriction, impeded respiration, asthma, and lung inflammation and allergies, among others, is of great practical importance. Such technology is clearly applicable to the treatment of heart, brain, lung, kidney, skin and other conditions, e.g. asthma and cancers such as leukemias, lymphomas, carcinomas, and the like, including colon cancer, breast cancer, lung cancer, liver cancer, prostate cancer, pancreatic cancer, hepatocellular carcinoma, kidney cancer, melanoma, etc., as well as all types of cancers which may metastasize or have metastasized, for instance, to the lung(s), breast, liver and prostate Similarly, a composition and method which are suitable for regular administration during a subject's daily routine, and that may be effectively administered preventatively, prophylactically and therapeutically, in conjunction with other therapies, or by itself for conditions without known therapies or as a substitute for therapies that have significant negative side effects is also of immediate clinical application.

Accordingly, there is still a need for improved treatments for respiratory, diseases associated with asthma, bronchoconstriction, and lung inflammation and allergies, whether or not accompanied by adenosine depletion, which are effective and easy to administer while being substantially non-toxic and cost effective.

### SUMMARY OF THE INVENTION

The present invention relates to the use of an epiandrosterone, analogues thereof, a ubiquinone, and/or pharmaceutically or veterinarily acceptable salts thereof, for the manufacture of a medicament for treating asthma and its associated symptoms.

The present invention also relates to a method of treating asthma and symptoms associated with it, such as bronchoconstriction, lung inflammation and allergies, and cancer in a subject in need of treatment by administering to the subject an epiandrosterone, an analog thereof, a ubiquinone, or a pharmaceutically or veterinarily acceptable salt thereof, in an amount effective to treat asthma or its associated symptoms.

The present invention relates to the use of folinic acid or a pharmaceutically or veterinarily acceptable salt thereof for the preparation of a medicament for treating adenosine depletion in a subject in need of such treatment, as set forth above.

The present invention also relates to a method of treating adenosine depletion in a subject in need of treatment, which comprises administering to the subject folinic acid or a pharmaceutically acceptable salt thereof in an amount effective to treat the adenosine depletion or to increase adenosine levels in certain tissues. The method may be applied to subjects afflicted with various diseases and conditions including steroid-induced adenosine depletion, anxiety, wasting disorder or weight loss, or subjects afflicted with any other disorder associated with adenosine depletion, or where an increase in adenosine levels would be therapeutically beneficial.

### DETAILED DESCRIPTION OF THE PREFERRED EMBODIMENTS

This invention arose from a desire of the inventor to provide never before available prophylactic and therapeutic treatments for certain respiratory and lung diseases and conditions, or treatments that are a substantial improvement over those presently available. The availability of a novel strategy to prevent and/or treat disorders and conditions associated with symptoms such as pulmonary bronchoconstriction, impeded respiration, lung inflammation and allergy(ies), among others, is of great practical importance.

Disclosed herein is a method of reducing adenosine levels, particularly in the lung, liver, heart and brain and, therefore, of treating asthma and its associated symptoms (bronchoconstriction, lung inflammation and allergies and difficult breathing), particularly non-steroid responding asthma, by administering to a subject in need of treatment an epiandrosterone (EA), analog thereof, a ubiquinone, or a pharmaceutically or veterinarily acceptable salt thereof, in an amount effective to inhibit or control asthma and/or its symptoms. Examples of EA and analogs thereof that may be used to carry out this method are represented by the chemical formula

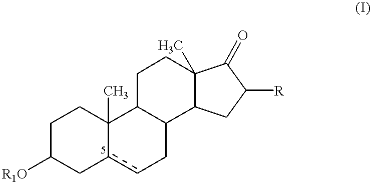

wherein the broken line represents an optional double bond; R comprises hydrogen or halogen; R₁ comprises hydrogen or SO₂OM, wherein M comprises H, Na, sulfatide

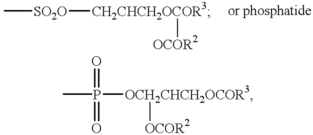

wherein R² and R^{3,} which may be the same or different, are straight or branched (C₁-C₁₄) alkyl or glucuronide

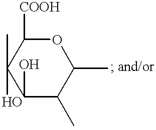

a ubiquinone or pharmaceutically or veterinarily acceptable salt thereof, wherein the ubiquinone has the chemical formula

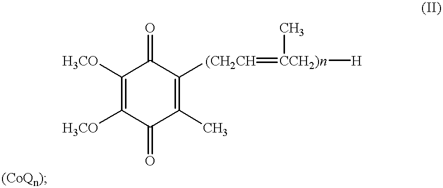

wherein n=1 to 12, the agent being present in an amount effective for altering levels of, or sensitivity to, adenosine in a subject's tissue (s), or treating bronchoconstriction, lung inflammation or allergies or a disease associated with either of them.

The hydrogen atom at position 5 of the formula (I) may be present in the alpha or beta configuration, or the compound may comprise a mixture of both configurations. Compounds illustrative of formula (I) above include DHEA, wherein R and R₁ are each hydrogen and the double bond is present; 16-alpha bromoepiandrosterone, where R is Br, R1 is H, and the double bond is present; 16-alpha-fluoro epiandrosterone, wherein R is F, R1 is H and double bond is present; Etiocholanolone, where R and R1 are each hydrogen and the double bond is absent; and Dehydroepiandrosterone sulphate, wherein R is H, R1 is SO2OM and M is a sulfatide group as defined above, and the double bond is absent, among others.

Preferably, in the compound of formula I, R may be halogen e.g., bromo, chloro, or fluoro), R1 comprises hydrogen, and the double bond is present. Most preferably, the compound of formula (I) comprises 16-alpha-fluoro epiandrosterone.

The compounds of formula (I) are made in accordance with known procedures, or variations thereof, that will be apparent to those skilled in the art. See, U.S. Pat. No. 4,956,355, UK Patent No. 2,240,472, EPO Patent Application No. 429,187, PCT Patent Publication WO 91/04030; see also M. Abou-Gharbia et al., J. Pharm. Sci. 70:1154-1157 (1981), Merck Index Monograph No. 7710, 11th Ed., (1989).

The ubiquinone may be administered by itself or concurrently with the DHEA or analog thereof in the methods of treating asthma described above. The phrase “concurrently administering” as used herein, means that the DHEA or the DHEA analog are administered either (a) simultaneously in time, preferably by formulating the two together in a common pharmaceutical carrier, or (b) at different times during the course of a common treatment schedule. In the latter case, the two compounds are administered at times sufficiently close for the ubiquinone to have as one of its effects to offset ubiquinone depletion in the subject's tissues, e. g. lungs and heart, and thereby counter-balance any deterioration of the tissue, e. g. lung and heart, function that may result from the administration of the DHEA or the analog thereof. The term “ubiquinone,” as used herein, refers to a family of compounds having structures based on a w,3-dimethoxy-5-methyl benzoquinone nucleus with a variable terpenoid acid chain containing one to twelve mono-unsaturated trans-isoprenoid units. Such compounds are known in the art as “Coenzyme Qₙ,” in which n equals 1 to 12. These compounds may be referred to herein as compounds represented by the formula

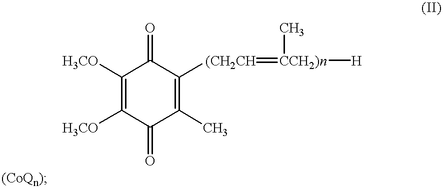

wherein n=1 to 10. Preferably, in the method of the invention, the ubiquinone is a compound according to formula given above, where n is 6 to 10, i.e., Coenzyme Q₆₋₁₀, and most preferably wherein n=10, i.e. Coenzyme Q₁₀.

Where the ubiquinone is formulated with a pharmaceutically acceptable carrier separately from the DHEA, analog thereof or salt thereof (e.g., where the DHEA, analog thereof or salt thereof is administered to the lungs of the subject, and the ubiquinone is administered systemically) it may be formulated by any of the techniques set forth above.

The compounds used to treat asthma, that is, the EA and ubiquinone or their salts, may be administered per se or in the form of pharmaceutically or veterinarily acceptable salts, as discussed above (the two together again being referred to as “active compounds”). The active compounds and their salts may be administered either systemically or topically, as discussed below. Generally, the ubiquinone is administered in an amount effective to increase ubiquinone levels, to offset ubiquinone depletion in the lungs and heart of the subject induced by the EA, analog thereof, or salt thereof, or to treat respiratory or lung symptoms of asthma, such as bronchoconstriction, lung inflammation and allergies, or cancer. The dosage will vary depending upon the condition of the subject and route of administration. The ubiquinone is preferably administered in a total amount per day of about 0.1, about 1, about 5, about 10, about 15, about 30 to about 50, about 100, about 150, about 300, about 600, about 900, about 1200 mg/kg body weight per day. More preferred are about 1 to about 150 mg/kg, about 30 to about 100 mg/kg, and most preferred about 5 to about 50 mg/kg. The ubiquinone may be administered once or several times a day. In general, the epiandrosterones and their salts are administered in a dosage of about 0.01, about 0.1, about 0.4, about 1, about 5, about 10, about 20 to about 4, about 30, about 70, about 100, about 300, about 600, about 1000, about 2000, about 3600 mg/kg body weight. The active compounds may be administered once or several times a day.

The method of treating adenosine depletion disclosed herein may be used to treat steroid-induced adenosine depletion, to stimulate adenosine synthesis, to treat or control anxiety, e.g., in treating premenstrual syndrome, to increase weight gain or treat wasting disorders, and to treat other adenosine-related pathologies, by administering folinic acid or its salts. Thus, the term “adenosine depletion” is intended to encompass conditions where adenosine levels are depleted in the subject as compared to previous adenosine levels in that subject, and conditions where adenosine levels are essentially the same as previous adenosine levels in that subject but, because of some other condition or alteration in that patient, a therapeutic benefit would be achieved in the patient by increased adenosine levels as compared to previous levels. The method is carried out preferably on patients where adenosine levels are depleted as compared to previous adenosine levels in the subject. The present invention is concerned primarily with the treatment of human subjects but may also be employed for the treatment of other mammalian subject, such as dogs and cats, for veterinary purpose.

Folinic acid and the pharmaceutically acceptable salts thereof, hereafter sometimes referred to as “active compounds”) are known, and may be made in accordance with known procedures. See, generally The Merck Index, Monograph No. 4141 (11th Ed. 1989); U.S. Pat. No. 2,741,608. Such pharmaceutically acceptable salts should be both pharmacologically and pharmaceutically or veterinarily acceptable and may be prepared as alkaline metal or alkaline earth salts, e.g., sodium, potassium or calcium salts, of the carboxylic acid group of folinic acid. The calcium salt of folinic acid is a preferred pharmaceutically or veterinarily acceptable salt.

The dosage of folinic acid or salt will vary depending on age, weight, and condition of the subject. Treatment may be initiated with small dosages less than optimum dose and increased until the optimum effect under the circumstances is reached. In general, the dosage will be from about 1, about 5, about 10 or about 20 mg/kg subject body weight, up to about 100, about 200, about 500, or about 1000 mg/kg subject body weight. Currently, dosages of from about 5 to about 500 mg/kg are preferred, dosages of from about 10 to about 200 mg/kg are more preferred, and dosages of from about 20 to about 100 mg/kg are most preferred. In general, the active compounds are preferably administered at a concentration that will afford effective results without causing any unduly harmful or deleterious side effects, and may be administered either as a single unit dose, or if desired in convenient subunits administered at suitable times throughout the day.

The active compounds are preferably administered to the subject as a pharmaceutical or veterinary composition. Pharmaceutical compositions for use in the present invention include those suitable for inhalation, and nasal, intrapulmonary, respirable, oral, topical (including buccal, sublingual, dermal and intraocular), parenteral (including subcutaneous, intradermal, intramuscular, intravenous and intraarticular), rectal, vaginal, implantable, and transdermal administration. The compositions may conveniently be presented in bulk or in single or multiple unit dosage forms and may be prepared by any of the methods that are well known in the art.

Compositions suitable for oral administration may be presented in discrete units, such as capsules, cachets, lozenges, or tablets, each containing a predetermined amount of the active compound; as a powder or granules; as a solution or a suspension in an aqueous or non-aqueous liquid; or as an oil-in-water or water-in-oil emulsion. Such compositions may be prepared by any suitable method of pharmacy that includes the step of bringing into association the active compound and a suitable carrier. In general, the compositions of the invention are prepared by uniformly and intimately admixing the active compound with a liquid or finely divided solid carrier, or both, and then, if necessary, shaping the resulting mixture. For example, a table may be prepared by compressing or molding a power or granules containing the active compound, optionally with one or more accessory ingredients. Compressed tablets may be prepared by compressing, in a suitable machine, the compound in a free-lowing form, such as a powder or granules optionally mixed with a binder, lubricant, inert diluent, and/or surface active/dispensing agent(s) or surfactant(s). Molded tablets may be made by molding, in a suitable machine, the powdered compound moistened with an inert liquid binder. Compositions for oral administration may optionally include enteric coatings known in the art to prevent degradation of the compositions in the stomach and provide release of the drug in the small intestine.

Compositions suitable for buccal or sub-lingual administration include lozenges comprising the active compound in a flavored base, usually sucrose and acacia or tragacanth, and pastilles comprising the compound in an inert base such as gelation and glycerin or sucrose and acacia.

Compositions suitable for parenteral administration comprise sterile aqueous and non-aqueous injection solutions, suspensions and emulsions of the active compound, which preparations are preferably isotonic with the blood of the intended recipient. These preparations may contain anti-oxidants, surfactants, buffers, bacteriostats, solutes that render the compositions isotonic with the blood of the intended recipient, and other formulation components known in the art. Aqueous and non-aqueous sterile suspensions may include suspending agents and thickening agents. The compositions may be presented in unit-dose or multi-dose containers, for example sealed ampoules and vials, and may be stored freeze-dried or lyophilized, requiring only the addition of the sterile liquid carrier, for example, saline or water-for-injection immediately prior to their use. Extemporaneous injection solutions, suspensions and emulsions may be prepared from sterile powders, granules and tablets of the kind previously described.

Compositions suitable for topical application to the skin preferably take the form of an ointment, cream, lotion, paste, gel, spray, aerosol, or oil, although other forms are also suitable. Carriers which may be used include vaseline, lanoline, polyethylene glycols, alcohols, transdermal enhancers, and combinations of two or more thereof.

Compositions suitable for rectal or vaginal administration are also included, and may be prepared by methods known in the art.

Compositions suitable for transdermal administration may be presented as discrete patches adapted to remain in intimate contact with the epidermis of the recipient for a prolonged period of time. Compositions suitable for transdermal administration may also be delivered by iontophoresis. See, e.g. Pharmaceutical Research 3:318 (1986), and typically take the form of an optionally buffered aqueous solution of the active compound.

The active compounds disclosed herein may be administered to the lungs of a subject by any suitable means, but are preferably administered by generating an aerosol or spray comprised of respirable, inhalable, nasal or intrapulmonarily delivered particles comprising the active compound, which particles the subject inhales, i.e. by inhalation administration. The respirable particles may be liquid or solid. Particles comprising the active compound for practicing the present invention should include particles of respirable or inhalable size; that is, particles of a size sufficiently small to pass through the mouth and larynx upon inhalation and into the bronchi and alveoli of the lungs. In general, particles ranging from about 0.05, about 0.1, about 0.5, about 1, about 1.5 to about 5, about 6, about 7, about 8, about 10 microns in size, more particularly particles about 0.5 to less than about 5 microns in size, are respirable or inhalable. When particles of nonrespirable size are included in the aerosol or spray, they tend to deposit in the throat and be swallowed. Thus, the quantity of non-respirable particles in the aerosol or spray is preferably minimized when intended for respirable administration or by inhalation. For nasal or intrapulmonary administration, a particle size in the range of about 10, about 11, about 15, about 20 to about 25, about 30, about 40, about 50, and sometimes even up to about 100 and about 500 microns is preferred to ensure retention in the nasal or pulmonary cavity. Pulmonary instillation is particularly useful for treating newborns.

Liquid pharmaceutical compositions of the active compound for producing an aerosol or spray may be prepared by combining the active compound with a stable vehicle, such as sterile pyrogen free water. Solid particulate compositions containing respirable dry particles of micronized active compound may be prepared by grinding dry active compound with a mortar and pestle, and then passing the micronized composition through a 400 mesh screen to break up or separate out large agglomerates. A solid particulate composition comprised of the active compound may optional contain a dispersant which serves to facilitate the formation of an aerosol. A suitable dispersant is lactose, which may be blended with the active compound in any suitable ratio, e.g. a 1 to 1 ratio by weight. Again, other therapeutic and formulation compounds may also be included, such as a surfactant to improve the sate of surfactant in the lung and help with the absorption of the active agent.

Aerosols of liquid particles comprising the active compound may be produced by any suitable means, such as with a nebulizer. See, e.g., U.S. Pat. No. 4,501,729. Nebulizers are commercially available devices which transform solutions or suspensions of the active ingredient into a therapeutic aerosol mist either by means of acceleration of a compressed gas, typically air or oxygen, through a narrow venturi orifice or by means of ultrasonic agitation. Suitable compositions for use in nebulizer consist of the active ingredient in liquid carrier, the active ingredient comprising up to 40% w/w of the compositions, but preferably less than 20% w/w he carrier is typically water or a dilute aqueous alcoholic solution, preferably made isotonic with body fluids by the addition of, for example sodium chloride. Optional additives include preservatives if the compositions is not prepared sterile, for example, methyl hydroxybenzoate, antioxidants, flavoring agents, volatile oils, buffering agents and surfactants.

Aerosols of solid particles comprising the active compound may likewise be produced with any sold particulate medicament aerosol generator. Aerosol generators for administering solid particulate medicaments to a subject product particles which are respirable, as explained above, and they generate a volume of aerosol containing a predetermined metered dose of a medicament at a rate suitable for human administration. Examples of such aerosol generators include metered dose inhalers and insufflators.

### EXAMPLES

The following examples are provided to more fully illustrate the present invention and should not be construed as restrictive thereof. In the examples provided below, EA means an epiandrosterone, DHEA means dehydroepiandrosterone, s means seconds, mg means milligrams, kg means kilograms, kw means kilowatts, Mhz means megahertz, CoQ means ubiquinone, and nmol means nanomoles.

### Examples 1 and 2

### Effects of Folinic Acid and DHEA on Adenosine Levels In Vivo

Young adult male Fischer 344 rats (120 grams) were administered dehydroepiandrosterone (DHEA) (300 mg/kg) or methyltestosterone (40 mg/kg) in carboxymethylcellulose by gavage once daily for fourteen days. Folinic acid (50 mg/kg) was administered intraperitoneally once daily for fourteen days. On the fifteenth day, the animals were sacrificed by microwave pulse (1.33 kw, 2450 MHZ, 6.5 s) to the cranium, which instantly denatures all brain protein and prevents further metabolism of adenosine. Hearts were removed from animals and flash frozen in liquid nitrogen with 10 seconds of death. Liver and lungs were removed en bloc and flash frozen with 30 seconds of death. Brain tissue was subsequently dissected. Tissue adenosine was extracted, derivatized to 1, N6-ethenoadenosine and analyzed by high performance liquid chromatography (HPLC) using spectrofluorometric detection according to the method of Clark and Dar (J. of Neuroscience Methods 25:243 (1988)). Results of these experiments are summarized in Table 1 below. Results are expressed as the mean±SEM, with κ p<0.05 compared to control group and φ p<0.05 compared to DHEA or methyltestosterone-treated groups.

TABLE 1
In Vivo Effect of DHEA, δ-1-methyltestosterone &
Folinic Acid on Adenosine Levels in Various Rat Tissues

| | Intracellular Adenosine | | |
|---|---|---|---|
| | (nmol/mg protein) | | |
| | Heart | Lung | Brain |
| Control | 10.6 ± 0.6 | 3.1 ± 0. | 0.5 ± 0.04 |
| | (n = 12) | (n = 6) | (n = 12) |
| DHEA | 6.7 ± 0.5 | 2.3 ± 0.3 | 0.19 ± 0.01 |
| (300 mg/kg) | (n = 12) | (n = 6) | (n = 12) |
| Methyltestosterone | 8.3 ± 1.0 | N.D. | 0.42 ± 0.06 |
| (40 mg/kg) | (n = 6) | | (n = 6) |
| Methyltestost. (M) | 6.0 ± 0.4 | ND. | 0.32 ± 0.03 |
| (120 mg/kg) | (n = 6) | | (n = 6) |
| Folinic Acid (F.A.) | 12.4 ± 2.1 | N.D. | 0.72 ± 0.09 |
| (50 mg/kg) | (n = 5) | | (n = 5) |
| DHEA + F.A. | 11.1 ± 0.6 | N.D. | 0.55 ± 0.09 |
| (300 mg/kg; 50 mg/kg) | (n = 5) | | (n = 5) |
| M + F.A. | 9.1 ± 0.4 | N.D. | 0.60 ± 0.06 |
| (120 mg/kg; 50 mg/kg) | (n = 6) | | (n = 6) |

N.D. = Not Determined

The results of these experiments indicate that rats administered DHEA or methyltestosterone daily for two weeks showed multi-organ depletion of adenosine. Depletion was dramatic in brain (60% depletion for DHEA, 34% for high dose methyltestosterone) and heart (37% depletion for DHEA, 22% depletion for high dose methyltestosterone). Co-administration of folinic acid completely abrogated steroid-mediated adenosine depletion. Folinic acid administered alone induce increase in adenosine levels for all organs studied.

### Example 3

### Effect of CoQs & an EA on In Vitro NADPH Levels

Glocose-6-Phosphate Dehydrogenase (G6PD) is an important enzyme that is widespread in mammals, and is involved in the conversion of NADP to NADPH, thereby increasing NADPH levels. An inhibition of the G6PD enzyme, thus, will be expected to result in a reduction of cellular NADPH levels, which event, in turn, will be expected to inhibit pathways that are heavily dependent on NADPH. One such pathway, the so-called One-Carbon-Pool pathway, also known as the Folate Pathway, is directly involved in the production of adenosine by addition of the C₂ and C₈ carbon atoms of the purine ring. Consequently, the inhibition of this pathway will lead to adenosine depletion.

The present invention is broadly applicable to Epiandrosterones (EAs) and Ubiquinones (CoQs). The description of the pathways involved in the present invention are described in the Background section. The present experiment was designed to show that one EA and two CoQs inhibit NADPH levels. DHEA, an Epiandrosterone, has already been shown to decrease levels of adenosine in various tissues. See, Examples 1 and 2 above. The fact that two CoQs are shown to lower NADPH levels to a similar extent as an Epiandrosterone, let alone to a similar extent ensures that the NADPH reduction caused by the CoQs will also result in lower cellular adenosine levels or in adenosine cell depletion. Thus, in accordance with the invention, both Epiandrosterones and Ubiquinones decrease levels of adenosine and, therefore, are useful as medicaments for use in the treatment of diseases where a decrease of adenosine levels or its depletion is desirable, including respiratory diseases such as asthma, bronchoconstriction, lung inflammation and allergies and the like. Both Ubiquinones and DHEA inhibit NADPH levels in a statistically significant manner, when compared to a control. Moreover, the Ubiquinone inhibits NADPH levels to a similar extent as DHEA. The present invention is broadly applicable to the use of Epiandrosterones (EAs) and Ubiquinones (CoQs) to the treatment of respiratory and lung diseases, and other diseases associated with varying levels of adenosine, adenosine hypersensitivity, asthma, bronchoconstriction, and/or lung inflammation and allergies. The DHEA and Ubiquinones employed in the present experiments are equivalent to those described and exemplified above.

Enzymatic Assay of Purified G6PDH

The reaction mixture contained 5OmM glycyl glycine buffer, pH 7.4, 2 mM D-glucose-6-phosphate, 0.67 mM Beta-NADP, 10 mM MgCL2 and 0.0125 units of G6PDH in a final volume of 3.0 ml. All experiments were repeated 4 times.

The control group contained 3 samples that were added no DHEA or Ubiquinone. The experimental group contained a similar number of samples (3) for each concentration of DHEA or Ubiquinone. One group was added DHEA (in triplicate) at different concentrations. A second group was added different concentrations of a CoQ of long side chain (in triplicate), and a third group received a CoQ of short side chain (in triplicate), both at various doses in the μM range.

The reaction was started by addition of the enzyme, and the increase in absorbance at 340 nm was measured for 5 minutes. Each data point was conducted in triplicate, and the full experiment was repeated 4 times.

Both DHEA and the Ubiquinones inhibited the enzyme activity in a statistically significant manner when compared to controls. DHEA was found to inhibit by 72% in vitro the activity of purified G6PDH when compared to control. Both Ubiquinones inhibited the activity of purified G6PDH in vitro by an amount that was not statistically significantly different from that of DHEA. Both DHEA and the Ubiquinones inhibited the enzyme in a statistically significant manner when compared to controls. Both long chain and short chain CoQs were found to be effective inhibitors of G6PDH.

The above results clearly indicate that CoQ reduced cellular levels of NADPH to an extent similar to DHEA and consequently cellular adenosine levels, and has a therapeutic effect on diseases and conditions associated with them. The present results show that CoQs have a therapeutic effect similar to that of epiandrosterones. The pathways involved in the present invention, as described above, show the criticality of the results reported here, showing that an Epiandrosterone (DHEA) and two Ubiquinones inhibit NADPH levels in a statistically significant manner. The same epiandrosterone (DHEA) was shown in Examples 1 and 2 to decrease levels of adenosine in various tissues. The two different Ubiquinones employed lowered NADPH levels to a similar extent as DHEA. The NADPH reduction caused by the Ubiquinones will, in the case of DHEA, result in lower cellular adenosine levels or adenosine depletion. Thus, in accordance with the invention, both Epiandrosterones and Ubiquinones decrease levels of adenosine and are, therefore, useful in the therapy of diseases and conditions where a decrease of adenosine levels or its depletion are desirable, including respiratory and airway diseases such as asthma, bronchoconstriction, lung inflammation and allergies, and the like.

These are clearly superior results, which could not have been expected based on the knowledge of the art at the time of this invention. The experimental data and results provided are clearly enabling of the effect of Ubiquinones on adenosine cellular levels and, therefore, on its therapeutic affect on diseases and conditions associated with them, as described and claimed in this patent.

The foregoing examples are illustrative of the present invention, and are not to be construed as limiting thereof. The invention is defined by the following claims, with equivalents of the claims to be included therein.

What is claimed as novel and unobvious in Letters Patent of the United States is:

1. A pharmaceutical or veterinary composition, comprising a carrier or diluent, and an active agent selected from
a dehydroepiandrosterone, or pharmaceutically or veterinarily acceptable salts thereof, the dehydroepiandrosterone having the chemical formula 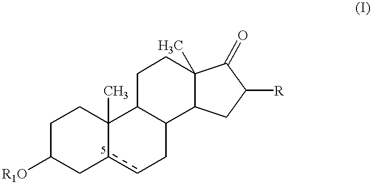
wherein the broken line represents a single or a double bond; R is hydrogen or a halogen; the H at position 5 is present in the alpha or beta configuration or the compound of chemical formula I comprises a racemic mixture of both configurations; and R¹ is hydrogen or SO₂OM, wherein M is selected from H, Na, sulfatide —SO₂O—CH₂CHCH₂OCOR³; or phosphatide 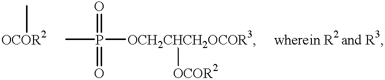
wherein R² and R³, which may be the same or different, are straight or branched (C₁-C₁₄) alkyl or glucuronide 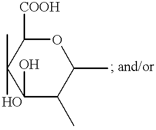
a ubiquinone or pharmaceutically or veterinarily acceptable salt thereof, wherein the ubiquinone has the chemical formula 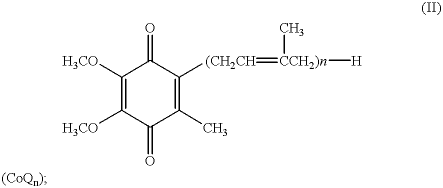
wherein n is 1 to 12, the agent being present in an amount effective for altering levels of, or sensitivity to, adenosine or adenosine receptors, adenosine in a subject's tissue (s), or treating bronchoconstriction, lung inflammation or allergies or a disease associated with either of them.

2. The composition of claim 1, wherein in the CoQₙ of formula II, wherein n is 1 to 10.

3. The composition of claim 1, wherein in the CoQₙ of formula II, wherein n is 6 to 10.

4. The composition of claim 3, wherein in the CoQₙ of formula II, wherein n is 10.

5. The composition of claim 4, comprising about 0.1 to about 40% w/w active agent.

6. The composition of claim 5, comprising about 1 to about 20% w/w active agent.

7. The composition of claim 1, wherein the compound of formula (I) is dehydroepiandrosterone, wherein R and R¹ are each hydrogen and the broken line represents a double bond.

8. The composition of claim 1, wherein the compound of formula (I) is 16-alpha bromoepiandrosterone, wherein R is Br, R¹ is H, and the broken line represents a double bond.

9. The composition of claim 1, wherein the compound of formula (I) is 16-alpha-fluoro epiandrosterone, wherein R is F, R¹ is H and the broken line represents a double bond.

10. The composition of claim 1, wherein the compound of formula (I) is etiocholanolone, wherein R and R¹ are each hydrogen and the broken line represents a double bond.

11. The composition of claim 1, wherein the compound of formula (I) is dehydroepiandrosterone sulfate, wherein R is H, R¹ is SO₂OM and M is a sulfatide group as defined above, and the broken line represents a single bond.

12. The composition of claim 1, wherein in the compound of formula (I), R is halogen selected from Br, Cl or F, R¹ is H, and the broken line represents a double bond.

13. The composition of claim 1, wherein the compound of formula (I) is 16-alpha-fluoro epiandrosterone.

14. The composition of claim 1, wherein the compound of formula (I) is selected from dehydroepiandrosterone, 16-alpha-bromoepiandrosterone, 16-alpha-fluoro epiandrosterone, etiocholanolone, dehydroepiandrosterone sulfate or pharmaceutically or veterinarily acceptable salts thereof.

15. The composition of claim 1, wherein the carrier or diluent comprises a pharmaceutically or veterinarily acceptable carrier or diluent.

16. The method of claim 15, wherein the carrier or diluent is selected from solid or liquid carriers or diluents, and the active agent comprises liquid or solid particles.

17. The composition of claim 15, further comprising an agent selected from folinic acid, pharmaceutically or veterinarily acceptable salts of folinic acid, other therapeutic agents, preservatives, antioxidants, flavoring agents, volatile oils, buffering agents, dispersants or surfactants.

18. The composition of claim 15, which is a systemic or topical formulation.

19. The formulation of claim 18, in the form of a formulation selected from buccal, sublingual, dermal, intraocular, vaginal, rectal, intraarticular, intrapulmonary respirable, oral, inhalable, nasal, topical, parenteral, or transdermal.

20. The formulation of claim 19, which is an oral formulation selected from the group consisting of capsules, cachets, lozenges, tablets, powder, granules, solutions, suspensions and emulsions.

21. The oral formulation of claim 19, which is a solution, suspension or emulsion selected from the group consisting of aqueous and non-aqueous liquid solutions and suspensions and oil-in-water and water-in-oil emulsions.

22. The oral formulation of claim 19, which is a buccal or sub-lingual formulation selected from the group consisting of lozenges further comprising a flavoring agent selected from the group consisting of sucrose, acacia and tragacanth; and pastilles further comprising an inert base selected from the group consisting of gelatin, glycerin, sucrose and acacia.

23. The oral formulation of claim 20, further comprising an enteric coating.

24. The formulation of claim 1, which is a parenteral formulation.

25. The parenteral formulation of claim 24, in injectable form.

26. The parenteral formulation of claim 24, selected from subcutaneous, intradermal, intramuscular, or intravenous formulations.

27. The injectable formulation of claim 24, selected from injectable solutions or suspensions, and which may further comprise folinic acid, pharmaceutically or veterinarily acceptable salts thereof, other therapeutic agents, antioxidants, buffers, bacteriostatic agents or solutes which render the solution or suspension isotonic with the blood of any intended recipient.

28. The injectable formulation of claim 27, wherein the solutions or suspensions are selected from sterile aqueous or non-aqueous injection solutions or suspensions, which may further comprise suspending agents or thickening agents.

29. The composition of claim 1 in bulk or in single or multi-dose form.

30. The composition of claim 29, wherein the single or multi-dose form is provided in sealed ampoules or vials.

31. The composition of claim 1, which is freeze-dried or lyophilized.

32. The formulation of claim 19, which is a topical formulation selected from ointments, creams, lotions, pastes, gels, sprays, aerosols or oils, which may further comprise a carrier selected from vaseline, lanoline, polyethylene glycols, alcohols or trans-dermal enhancers.

33. The formulation of claim 19, which is a transdermal formulation in the form of a patch.

34. The transdermal formulation of claim 33, which is an iontophoretic formulation selected from iontophoretic solutions or suspensions, and which may further comprise a buffer.

35. The formulation of claim 19, which is an inhalable, respirable, intrapulmonary or nasal formulation.

36. The inhalable or respirable formulation of claim 35, which is an aerosol or spray comprising liquid or solid particles of the active agent, and which may further comprise an ingredient selected from folinic acid, other therapeutic agents, preservatives, antioxidants, flavoring agents, volatile oils, buffering agents, dispersants or surfactants.

37. The formulation of claim 36, comprising an inhalable or respirable formulation comprising powdered or liquid particles of the active agent about 0.05 to about 10μ in size.

38. The formulation of claim 37, comprising an inhalable or respirable aerosol formulation comprising powdered or liquid particles of the active agent about 0.1 to 5μ in size.

39. The formulation of claim 36, which comprises a nasal or intrapulmonary aerosol formulation comprising powdered or liquid particles of the active agent about 10 to about 100μ in size.

40. The formulation of claim 39, which comprises powdered or liquid particles of the active agent about 10 to 50μ in size.

41. The formulation of claim 16, wherein the carrier comprises a hydrophobic carrier.

42. A kit comprising the formulation of claim 15, and a delivery device.

43. The kit of claim 42, wherein the formulation comprises an inhalable, respirable, intrapulmonary or nasal formulation, and the delivery device comprises an inhaler provided with an aerosol generating means.

44. The kit of claim 42, wherein the delivery device delivers individual pre-metered doses of the formulation.

45. The kit of claim 42, wherein the delivery device comprises an inhaler.

46. The kit of claim 42, wherein the inhaler comprises a nebulizer or insufflator.

47. The kit of claim 42, wherein the delivery device comprises a compression inhaler, and the formulation comprises a suspension or solution in an aqueous or non-aqueous liquid or an oil-in-water or water-in-oil emulsion.

48. The kit of claim 41, wherein the formulation is provided in a pierceable or openable capsule or cartridge.

49. An in vivo method of preventing or treating a disorder or condition associated with abnormal levels of adenosine or adenosine receptors, or sensitivity to adenosine in a subject's tissue(s), bronchoconstriction, lung inflammation or allergies, wheezing, difficult breathing, impeded airways, asthma, COPD, CF, ARDS, RDS, decreased lung surfactant, pulmonary fibrosis, allergic rhinitis, or cancer, comprising administering to a subject in need of treatment a therapeutic amount of the active composition of claim 1, wherein when the epiandrosterone is the sole agent and the disease or condition is steroid induced asthma, the composition may not comprise a steroid.

50. The method of claim 49, wherein the disorder or condition is associated with bronchoconstriction, impeded respiration or wheezing.

51. The method of claim 49, wherein the active agent comprises a dehydroepiandrosterone of chemical formula (I) or its salt.

52. The method of claim 51, wherein the epiandrosterone salt comprises dehydroepiandrosterone sulfate.

53. The method of claim 51, wherein the active agent comprise dehydroepiandrosterone (DHEA).

54. The method of claim 49, wherein epiandrosterone or salt thereof, is administered in an amount of about 0.05 to about 1,000 mg/kg body weight.

55. The method of claim 51, wherein the epiandrosterone or salt thereof is administered in an amount of about 1 to about 600 mg/kg body weight.

56. The method of claim 51, wherein the epiandrosterone or salt thereof is administered in an amount of about 5 to about 200 mg/kg body weight.

57. The method of claim 49, wherein the active agent is a ubiquinone of chemical formula (II) or salt thereof.

58. The method of claim 57, wherein the ubiquinone or salt thereof is administered in an amount of about 1 to about 1200 mg/kg body weight.

59. The method of claim 58 herein the ubiquinone or salt thereof is administered in an amount of about 30 to about 600 mg/kg body weight.

60. The method of claim 59, wherein the ubiquinone or salt thereof is administered in an amount of about 50 to about 150 mg/kg.

61. The method of claim 49, further comprising administering folinic acid or a salt thereof.

62. The method of claim 61, wherein the folinic acid is administered in an amount about 1 to about 1,000 mg/kg body weight.

63. The method of claim 62, wherein the folinic acid is administered in an amount of about 5 to about 500 mg/kg body weight.

64. The method of claim 49, wherein the disorder or condition is chronic obstructive pulmonary disease (COPD).

65. The method of claim 49, wherein the disorder or condition is acute respiratory distress syndrome (ARDS).

66. The method of claim 49, wherein the disorder or condition comprises lung inflammation or allergy(ies).

67. The method of claim 49, wherein the disorder or condition comprises pulmonary fibrosis.

68. The method of claim 49, wherein the disorder or condition comprises allergic rhinitis.

69. The method of claim 49, wherein the disorder or condition comprises infantile respiratory distress syndrome (RDS).

70. The method of claim 49, wherein the disorder or condition comprises cystic fibrosis (CF).

71. The method of claim 49, wherein the disorder or condition comprises impeded respiration or lung pain.

72. The method of claim 49, wherein the disorder or condition comprises decreased lung surfactant.

73. The method of claim 49, wherein the disorder or condition comprises lung cancer.

74. The method of claim 49, wherein the active agent comprises a ubiquinone and an epiandrosterone or their salts, and they are administered concurrently.

75. The method of claim 74, wherein the epiandrosterone and the ubiquinone or their salts are administered in the same formulation.

76. The method of claim 74, wherein the epiandrosterone and the ubiquinone or their salts are administered in different formulations.

77. The method of claim 49, wherein the subject is a human or non-human animal.

78. The method of claim 49, which is a prophylactic or therapeutic method.

79. The method of claim 49, wherein the subject is in need of treatment to reduce or deplete adenosine levels and/or increase ubiquinone levels.
